(19) United States
(12) Patent Application Publication
(10) Pub. No.: US 2011/0262398 A1
(43) Pub. Date: Oct. 27, 2011

(54) CARDIAC TREATMENT USING ANTI-FIBROTIC AGENTS

(75) Inventors: Samuel C. Dudley, Chicago, IL (US); Roy L. Sutliff, Decatur, GA (US)

(73) Assignee: UNIVERSITY OF ILLINOIS, Chicago, IL (US)

(21) Appl. No.: 13/091,972

(22) Filed: Apr. 21, 2011

Related U.S. Application Data
(60) Provisional application No. 61/326,300, filed on Apr. 21, 2010.

(51) Int. Cl. A61K 38/21; A61P 9/00; A61K 38/00; A61K 31/52; A61K 31/66

(52) U.S. Cl. 424/85.5; 514/263.2; 514/110; 514/16.4

(57) ABSTRACT

Disclosed herein are reagents and methods for detecting and treating age-related diastolic dysfunction in an animal or a human.

This application is related to and claims priority to U.S. provisional patent application, Ser. No. 61/326,300, filed Apr. 21, 2010, the entirety of the contents thereof being expressly incorporated by reference herein.

This invention was made with government support under grants R01 HL085558, HL073753 and HL058000 awarded by the National Institutes of Health. The government has certain rights in the invention.

### BACKGROUND OF THE INVENTION

1. Field of the Invention

This invention is related to heart disease in the elderly and aged, particularly heart disease characterized by diastolic dysfunction in the absence of systolic dysfunction (i.e., heart failure with preserved ventricular function). The invention provides reagents and methods for identifying diastolic dysfunction in elderly patients related to fibrosis in the heart muscle, particularly fibrosis mediated by increased expression, activity or both of cytokines that increase expression of extracellular matrix components. Also provided are reagents and methods for ameliorating the effects of such cytokines and reducing fibrosis and fibrotic pathologies associated with age-related diastolic dysfunction.

2. Background of the Related Art

Heart failure is a major and growing public health concern in the United States; there are an estimated 5 million people living with this disease, and another 550,000 patients will be diagnosed yearly (Brutsaert, 2003, Cardiac endothelial-myocardial signaling: its role in cardiac growth, contractile performance, and rhythmicity. Physiol Rev 83: 59-115). Approximately half of all heart failure patients in the United States suffer from diastolic heart failure, and it is a major cause of mortality in the elderly population (Zile & Brutsaert, 2002, New concepts in diastolic dysfunction and diastolic heart failure: Part I: diagnosis, prognosis, and measurements of diastolic function. Circulation 105: 1387-1393), Diastolic heart failure describes a group of patients whose clinical manifestation of congestive heart failure is characterized by normal left ventricular diastolic volume, a normal ejection fraction, delayed active relaxation, and increased passive stiffness of the left ventricle (Zile et al., 2005, Diastolic heart failure: definitions and terminology. Prog Cardiovasc Dis 47: 307-313). Diastolic dysfunction precedes diastolic heart failure and is often clinically silent; it is characterized by abnormal ventricular distensibility, relaxation, and filling (Aurigemma & Gaasch, 2004, Clinical practice. Diastolic heart failure. N Engl J Med 351: 1097-1105). Both diastolic dysfunction and diastolic heart failure are most common in the elderly population. In a study that examined the prevalence of this form of heart failure, 50% of patients over the age of 70 showed evidence of diastolic heart failure. Studies indicate that the most important determinants for development of diastolic dysfunction and diastolic heart failure are age and hypertension (Zile & Brutsaert, 2002, Id.). Although the exact molecular mechanisms behind diastolic dysfunction are poorly understood, fibrosis is thought to contribute to its progression. Increased activity of cytokines and accumulation of extracellular matrix proteins are key features of most fibrotic diseases (Leask, 2007, TGFbeta, cardiac fibroblasts, and the fibrotic response. Cardiovasc Res 74: 207-212). Transforming growth factor beta (TGF-β), a pro-fibrotic cytokine, works synergistically with connective tissue growth factor (CTGF) to promote fibroblast proliferation and deposition of collagen and fibronectin (Lim & Zhu, 2006, Role of transforming growth factor-beta in the progression of heart failure. Cell Mol Life Sci 63: 2584-2596).

While well-established murine models exist to study such common cardiovascular diseases as hypertension, atherosclerosis, and congestive heart failure, a model of isolated age-related diastolic dysfunction has yet to be established and characterized. Previous studies have established that the ratio of early to late mitral filling velocity is decreased in aged wild-type mice (27) and that caloric restriction improves diastolic function in aged mice (28), but there has not been a model of pure spontaneous diastolic dysfunction or a model that has established the connection between age-related diastolic dysfunction and fibrosis.

Thus there is a need in this art to identify the role of fibrosis in the development of age-related diastolic dysfunction and to develop reagents and methods for detecting and treating diastolic dysfunction, particularly in elderly patients.

### SUMMARY OF THE INVENTION

This invention provides reagents and methods for detecting and treating diastolic dysfunction, particularly in elderly patients.

In a first aspect, the invention provides methods for detecting age-associated diastolic dysfunction in a human or animal, comprising the steps of identifying abnormal production or proliferation of extracellular matrix components or increased expression of a cytokine that produces or facilitates abnormal proliferation of extracellular matrix components. In certain embodiments, the abnormal proliferation of extracellular matrix components produces cardiac fibrosis that can be detected in heart of a human or animal with age-associated diastolic dysfunction. In certain embodiments, the human or animal exhibits myocardial or left or right ventricular diastolic stiffness. In certain embodiments, the abnormally produced or proliferated extracellular matrix components include but are not limited to various collagen species (including but not limited to collagen 1A1 and collagen 3A, fibronectin elastin, laminin, and alpha smooth-muscle actin (α-SMA), as well as proteoglycans, heparin sulfate, chondroitin sulfate, keratin sulfate, and hyaluronic acid). In certain embodiments, cytokines that produce or facilitate abnormal proliferation of extracellular matrix components by increased expression thereof include but are not limited to transforming growth factor-β (TGF-β), connective tissue growth factor (CTGF), or a SMAD protein. In particular embodiments, the human or animal does not exhibit significant hypertension.

In a second aspect, the invention provides methods for treating age-associated diastolic dysfunction in a human or animal, comprising the steps of administering an anti-fibrotic agent to a human or animal that has abnormal production or proliferation of extracellular matrix components or increased expression of a cytokine that produces or facilitates abnormal proliferation of extracellular matrix components. In certain embodiments, the abnormal proliferation of extracellular matrix components produces cardiac fibrosis that can be detected in heart of a human or animal with age-associated diastolic dysfunction. In certain embodiments, human or animal exhibits myocardial or left or right ventricular diastolic stiffness. In certain embodiments, the abnormally produced or proliferated extracellular matrix components include but are not limited to various collagen species (including but not limited to collagen 1A1 and collagen 3A, fibronectin elastin, laminin, and alpha smooth-muscle actin (α-SMA), as well as proteoglycans, heparin sulfate, chondroitin sulfate, keratin sulfate, and hyaluronic acid. In certain embodiments, cytokines that produce or facilitate abnormal proliferation of extracellular matrix components by increased expression thereof include but are not limited to transforming growth factor-β (TGF-β), connective tissue growth factor (CTGF), or a SMAD protein. In certain embodiments, age-associated diastolic dysfunction is treated according to the methods of the invention by administering a therapeutically effective amount of at least one of a transforming growth factor-β (TGF-β) inhibitor, a connective tissue growth factor (CTGF) inhibitor, or a SMAD protein inhibitor. In particular embodiments, the human or animal does not exhibit significant hypertension or oxidative stress.

In a third aspect, the invention provides methods for treating age-associated diastolic dysfunction in a human or animal in the absence of systolic dysfunction, comprising the steps of administering an anti-fibrotic agent to a human or animal that has abnormal production or proliferation of extracellular matrix components or increased expression of a cytokine that produces or facilitates abnormal proliferation of extracellular matrix components. In certain embodiments, the abnormal proliferation of extracellular matrix components produces cardiac fibrosis that can be detected in heart of a human or animal with age-associated diastolic dysfunction. In certain embodiments, human or animal exhibits myocardial or left or right ventricular diastolic stiffness. In certain embodiments, the abnormally produced or proliferated extracellular matrix components include but are not limited to various collagen species (including but not limited to collagen 1A1 and collagen 3A, fibronectin elastin, laminin, and alpha smooth-muscle actin (α-SMA), as well as proteoglycans, heparin sulfate, chondroitin sulfate, keratin sulfate, and hyaluronic acid). In certain embodiments, cytokines that produce or facilitate abnormal proliferation of extracellular matrix components by increased expression thereof include but are not limited to transforming growth factor-β (TGF-β), connective tissue growth factor (CTGF), or a SMAD protein. In certain embodiments, age-associated diastolic dysfunction is treated according to the methods of the invention by administering a therapeutically effective amount of at least one of a transforming growth factor-β (TGF-β) inhibitor, a connective tissue growth factor (CTGF) inhibitor, or a SMAD protein inhibitor. In particular embodiments, the human or animal does not exhibit significant hypertension or oxidative stress.

In a fourth aspect, the invention provides pharmaceutical compositions comprising at least one of an inhibitor of transforming growth factor-β (TGF-β), an inhibitor of connective tissue growth factor (CTGF), or an inhibitor of a SMAD protein and a pharmaceutically acceptable carrier, diluent or excipient. Preferred embodiments of said at least one of an inhibitor of transforming growth factor-β (TGF-β), an inhibitor of connective tissue growth factor (CTGF), or an inhibitor of a SMAD protein excludes human growth hormone or antibodies immunologically specific for TGF-β.

In a fifth aspect, the invention provides kits comprising a pharmaceutical composition comprising at least one of an inhibitor of transforming growth factor-β (TGF-β), an inhibitor of connective tissue growth factor (CTGF), or an inhibitor of a SMAD protein and a pharmaceutically acceptable carrier, diluent or excipient and optionally instructions for the use thereof. In certain embodiments, kits of the invention provide said pharmaceutical compositions in unit dosage forms. In certain embodiments, kits of the invention provide the at least one of an inhibitor of transforming growth factor-β (TGF-β), an inhibitor of connective tissue growth factor (CTGF), or an inhibitor of a SMAD protein in a form, such as a dried or lyophilize form, stable for storage at ambient or reduced temperature, and liquid or other vehicle for preparing the pharmaceutical composition comprising a pharmaceutically acceptable carrier, diluent or excipient.

Specific preferred embodiments of the invention will become evident from the following more detailed description of certain preferred embodiments and the claims.

### BRIEF DESCRIPTION OF THE DRAWINGS

The invention as set forth herein can be further appreciated with reference to the drawings. Data as set forth herein are represented as the mean ± standard error and are compared using the Student's t-test when SAMR1 and SAMP8 mice are being compared for 6 month time point studies. A two-way ANOVA is used when SAMR1 and SAMP8 mice are being compared for 3- and 6-month studies. Bonferroni post-hoc tests were used to determine significance of specific pair-wise comparisons. A p value <0.05 is considered statistically significant.

FIG. 1 depicts histograms showing increased expression of p19, a marker of senescence, in SAMP8 mice showing accelerated senescence at 6 months of age. p19 expression was measured by quantitative RT-PCR, and p19 copy number is normalized to 18S copy number (n=7, *p<0.05).

FIG. 2 is a histogram of mean arterial pressure was unchanged in SAMR1 and SAMP8 mice from 3 to 6 months of age (n=5, p=ns).

FIGS. 3A through 3D are histograms illustrating functional analysis of isolated cardiomyocytes. FIG. 3A is a histogram showing that the mean of diastolic sarcomere length was significantly shorter in cardiomyocytes from SAMP8 compared to SAMR1 at 6 month old age (n=53, 59, *p<0.05). FIG. 3B is a histogram showing fractional shortening of isolated cardiomyocytes paced at 1.0 Hz at 37° C. represented as the peak shortening divided by the baseline sarcomere length (n=53, 59, p=NS). FIG. 3C is a histogram showing times to 90% peak contraction in isolated cardiomyocytes (n=47, 51, p=NS). FIG. 3D is a histogram showing isolated cardiomyocytes from SAMP8 mice have a prolonged relaxation constant (τ) compared to SAMP1 mice (n=53, 59, p=NS).

FIGS. 4A and 4B show the results of collagen-specific staining in heart muscle in SAM mice. FIG. 4A are photomicrographs of picrosirius red staining to evaluate collagen deposition. Using bright-field microscopy, SAMP8 mice show a more intense red stain than SAMR1 mice, indicating greater collagen accumulation. When polarized light is used, larger collagen fibers appear as bright yellow or orange, and thinner fibers are green. Both modalities show increased collagen accumulation in SAMP8 mice at 6 months of age and hence increased cardiac fibrosis in both the interstitial areas and perivascular areas compared to SAMR1 controls at 6 months of age. Similar results are shown in FIG. 4B using Masson's trichrome staining to evaluate collagen deposition (n=4, *p<0.01). FIG. 4B also shows histograms showing % collagen specific staining in interstitial fibrosis and perivascular fibrosis.

FIGS. 5A through 5C are histograms of comparative expression of collagen 1A1 (FIG. 5A), collagen 3A (FIG. 5B) and fibronectin (FIG. 5C) in SAMR1 and SAMP8 mice relative to 18S RNA expression determined by quantitative real-time PCR. SAMP8 mice showed increased gene expression of collagens 1A1 and 3A and as well as fibronectin 1 compared to SAMR1 controls at 6 months of age (n=7, *p<0.05).

FIGS. 6A through 6C are histograms of comparative expression of TGF-β (FIG. 6A), connective tissue growth factor (CTGF) (FIG. 6B) and alpha smooth-muscle actin (α-SMA) (FIG. 6C) in SAMR1 and SAMP8 mice relative to 18S RNA expression determined by quantitative real-time PCR. SAMP8 mice show increased gene expression of TGF-β compared to SAMR1 controls at 6 months of age. (n=7, *p<0.05).

### DETAILED DESCRIPTION OF PREFERRED EMBODIMENTS

This invention provides reagents and methods for identifying age-associated diastolic dysfunction in an animal and particularly a human, most particularly an aged or elderly human. The methods of this invention include detecting increased or abnormal production or proliferation of extracellular matrix components in the heart, as well as increased or abnormal production of cytokines that stimulate increased or abnormal production or proliferation of extracellular matrix components in the heart. The invention also provides reagents, particularly inhibitors of said cytokine expression, for administering to an animal and particularly a human, most particularly an aged or elderly human to ameliorate the effects of age-associated diastolic dysfunction in the human. The reagents and methods of the invention are particularly directed to said age-associated diastolic dysfunction that is not accompanied by significant hypertension in the animal or human.

As used herein, the terms “patient,” “mammal” and “animal” includes human and animal subjects.

As used herein, “treatment” or “treat” refers to both therapeutic treatment and prophylactic or preventative measures. Those in need of treatment include those already having an immunological disorder as well as those prone to have the disorder or those in which the disorder is to be prevented.

As used herein, the term “age-associated diastolic dysfunction” is intended to encompass age-related changes in the heart muscle, particularly the ventricles and most particularly the left ventricle, that result in impaired relaxation of the heart muscle. Specifically, the term describes conditions wherein relaxation of the heart muscle is impaired, resulting in increased ventricular pressure with pulmonary (congestion) as well as systemic (increased back pressure from the vasculature) consequences, including inter alia congestive heart failure.

As used herein, the term “cardiac fibrosis” is intended to encompass stiffening of the heart muscle, particularly relating to the cardiac interstitium and associated with age and generally due to abnormal production of extracellular matrix proteins, including various collagens, fibronectin and other associated proteins.

As used herein, the term “oxidative stress” is intended to encompass cellular conditions of excess reactive oxygen species, either by increased production thereof or reduction in production of antioxidants such as glutathione.

As used herein, the term “extracellular matrix component” is intended to encompass various collagen species (including but not limited to collagen 1A1 and collagen 3A, fibronectin elastin, laminin, and alpha smooth-muscle actin (α-SMA), as well as proteoglycans, heparin sulfate, chondroitin sulfate, keratin sulfate, and hyaluronic acid).

As used herein, the term “anti-fibrotic agent” includes but is not limited to azathioprine and cyclophosphamide, IFN-gamma, certain lectins (as disclosed, for example. in U.S. Pat. No. 7,026.287), decorin, LPA1 receptor inhibitors, AM966 (available from Amira Pharmaceuticals, Inc., San Diego, Calif.), GC1008 (available from Genzyme, Cambridge, Mass.), and FG-3019 (available from FibroGen, Inc., San Francisco, Calif.).

As used herein, the term “TGF-β inhibitor” is intended to encompass naturally occurring inhibitors, including but not limited to chordin and noggin proteins, Cerebus, Gremlin, DAN and other members of the DAN protein family, and follistatin, as well as synthetic inhibitors, including but not limited to SB43154 (GlaxoSmithKline, King of Prussia, Pa.), LY 2157299 (Axon Medichem, Groningen, NL) and TGF-β receptor kinase inhibitors (TβRI-KI) such as [3-(pyridine-2yl)-4-(4-quinonyl)]-1H pyrazole and SD-208 (Scios, Inc, Fremont, Calif.), or TGF-beta 2 inhibitor AP 12009 (Antisense Pharma, Regensburg, Bavaria), Pirfenidone (InterMune Inc., Brisbane, Calif.), or as set forth in Yingling et al., 2004, Nature Reviews Drug Discovery 3, 1011-1022.

As used herein, the term “connective tissue growth factor inhibitor” is intended to encompass synthetic inhibitors, including but not limited to I,5-dihydro-7-(1-piperidinyl)imidazo[2,1-b]quinazolin-2(3H)-one dihydrochloride hydrate, DN-9693 (Tanaka et al., 1987, J. Pharm. Sci. 76: 235-237), UK-156406 (Pfizer Central Research, Sandwich, Kent, UK, statins, including simvastatin and lovastatin, as well as naturally occurring CTGF inhibitors including but not limited to interleukin-1α (Nowinski et al., 2002, Journal of Investigative Dermatology 119: 449-455), perlecan, caffeine, paraxanthine, and EXC 001 (Excalliard Pharmaceuticals, Inc., Carlsbad, Calif.).

The methods provided herein can be practiced according to generally accepted principles in the medical arts, relating to identifying abnormal (i.e. increased) expression of extracellular matrix components or increased expression cytokines associated with stimulating expression of extracellular matrix components. Said methods can be employed to detect abnormal (i.e. increased) expression of extracellular matrix components or increased expression cytokines associated with stimulating expression of extracellular matrix components. In certain embodiments, such methods include non-invasive imaging such as cardiac magnetic resonance imaging. In additional embodiments, methods using biopsied samples can be employed including detecting increased mRNA or protein expression, immunohistochemistry, nucleic acid hybridization, and various embodiments of the polymerase chain reaction (including but not limited to reverse transcriptase PCR, quantitative PCR and real-time quantitative PCR) and similar methods know to those with skill in the arts. Methods wherein abnormal (i.e. increased) expression of extracellular matrix components or increased expression cytokines associated with stimulating expression of extracellular matrix components are treated by reversing, reducing or inhibiting said abnormal expression comprise generally administering inhibitors of said abnormal expression to a human or animal, particularly as pharmaceutical compositions as set forth herein or in any medically acceptable form that is therapeutically effective. As used herein, the term “SMAD protein inhibitor” is intended to encompass

In certain embodiments the invention provides pharmaceutical compositions comprising at least one of an inhibitor of transforming growth factor-β (TGF-β), an inhibitor of connective tissue growth factor (CTGF), or an inhibitor of a SMAD protein and a pharmaceutically acceptable diluent, carrier, solubilizer, emulsifier, preservative and/or adjuvant, wherein the pharmaceutical composition is capable of inducing a desired therapeutic effect when properly administered to a patient. Preferably, acceptable formulation materials are nontoxic to recipients at the dosages and concentrations employed.

The expression “therapeutically effective” in reference to a pharmaceutical composition comprising at least one of an inhibitor of transforming growth factor-β (TGF-β), an inhibitor of connective tissue growth factor (CTGF), or an inhibitor of a SMAD protein is understood to mean, according to the invention, an amount of the said pharmaceutical composition that is capable of preventing, ameliorating or reducing the pathological effects induced by abnormal expression or proliferation of extracellular matrix components, including cardiac fibrosis associated with age-associated diastolic dysfunction. For example, a pharmaceutical composition is therapeutically effective where a patient who has age-associated diastolic dysfunction has less severe or reduced symptoms when treated with the pharmaceutical composition compared with symptoms prior to said treatment. A pharmaceutical composition administered to a patient is also therapeutically effective where symptoms associated with age-associated diastolic dysfunction are prevented from occurring in a patient who has a history of such symptoms or who is considered likely to present with such symptoms, or is particularly at risk to develop diastolic dysfunction from genetic, familial, environmental or behavioral factors.

In certain embodiments, a pharmaceutical composition useful in the methods of the invention may contain formulation materials for modifying, maintaining or preserving, for example, the pH, osmolarity, viscosity, clarity, color, isotonicity, odor, sterility, stability, rate of dissolution or release, adsorption or penetration of the composition. In such embodiments, suitable formulation materials include, but are not limited to, amino acids (such as glycine, glutamine, asparagine, arginine or lysine); antimicrobials; antioxidants (such as ascorbic acid, sodium sulfite or sodium hydrogen-sulfite); buffers (such as borate, bicarbonate, Tris-HCl, citrates, phosphates or other organic acids); bulking agents (such as mannitol or glycine); chelating agents (such as ethylenediamine tetraacetic acid (EDTA)); complexing agents (such as caffeine, polyvinylpyrrolidone, beta-cyclodextrin or hydroxypropyl-beta-cyclodextrin); fillers; monosaccharides; disaccharides; and other carbohydrates (such as glucose, mannose or dextrins); proteins (such as serum albumin, gelatin or immunoglobulins); coloring, flavoring and diluting agents; emulsifying agents; hydrophilic polymers (such as polyvinylpyrrolidone); low molecular weight polypeptides; salt-forming counterions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate 20, polysorbate 80, triton, tromethamine, lecithin, cholesterol, tyloxapal); stability enhancing agents (such as sucrose or sorbitol); tonicity enhancing agents (such as alkali metal halides, preferably sodium or potassium chloride, mannitol sorbitol); delivery vehicles; diluents; excipients and/or pharmaceutical adjuvants. See REMINGTON'S PHARMACEUTICAL SCIENCES, 18^{th} Edition, (A. R. Gennaro, ed.), 1990, Mack Publishing Company.

In certain embodiments, the optimal pharmaceutical composition will be determined by one skilled in the art depending upon, for example, the intended route of administration, delivery format and desired dosage. See, for example, REMINGTON'S PHARMACEUTICAL SCIENCES, supra. In certain embodiments, such compositions may influence the physical state, stability, rate of in vivo release and rate of in vivo clearance of the at least one of an inhibitor of transforming growth factor-β (TGF-β), an inhibitor of connective tissue growth factor (CTGF), or an inhibitor of a SMAD protein as provided in a pharmaceutical composition of the invention.

In certain embodiments, the primary vehicle or carrier in a pharmaceutical composition may be either aqueous or non-aqueous in nature. For example, a suitable vehicle or carrier may be water for injection, physiological saline solution or artificial cerebrospinal fluid, possibly supplemented with other materials common in compositions for parenteral administration. Neutral buffered saline or saline mixed with serum albumin are further exemplary vehicles. In preferred embodiments, pharmaceutical compositions of the present invention comprise Tris buffer of about pH 7.0-8.5, or acetate buffer of about pH 4.0-5.5, and may further include sorbitol, sucrose, Tween-20 and/or a suitable substitute therefor. In certain embodiments of the invention, compositions of the at least one of an inhibitor of transforming growth factor-β (TGF-β), an inhibitor of connective tissue growth factor (CTGF), or an inhibitor of a SMAD protein may be prepared for storage by mixing the selected composition having the desired degree of purity with optional formulation agents (REMINGTON'S PHARMACEUTICAL SCIENCES, supra) in the form of a lyophilized cake or an aqueous solution. Further, in certain embodiments, the product comprising the at least one of an inhibitor of transforming growth factor-β (TGF-β), an inhibitor of connective tissue growth factor (CTGF), or an inhibitor of a SMAD protein and a pharmaceutically may be formulated as a lyophilizate using appropriate excipients such as sucrose.

The pharmaceutical compositions of the invention can be selected for parenteral delivery. Alternatively, the compositions may be selected for inhalation or for delivery through the digestive tract, such as orally. Preparation of such pharmaceutically acceptable compositions is within the skill of the art.

The formulation components are present preferably in concentrations that are acceptable to the site of administration. In certain embodiments, buffers are used to maintain the composition at physiological pH or at a slightly lower pH, typically within a pH range of from about 5 to about 8.

When parenteral administration is contemplated, the therapeutic compositions for use in this invention may be provided in the form of a pyrogen-free, parenterally acceptable aqueous solution comprising the desired at least one of an inhibitor of transforming growth factor-β (TGF-β), an inhibitor of connective tissue growth factor (CTGF), or an inhibitor of a SMAD protein in a pharmaceutically acceptable vehicle. A particularly suitable vehicle for parenteral injection is sterile distilled water in which the at least one of an inhibitor of transforming growth factor-β (TGF-β), an inhibitor of connective tissue growth factor (CTGF), or an inhibitor of a SMAD protein is formulated as a sterile, isotonic solution, properly preserved. In certain embodiments, the preparation can involve the formulation of the desired molecule with an agent, such as injectable microspheres, bio-erodible particles, polymeric compounds (such as polylactic acid or polyglycolic acid), beads or liposomes, that may provide controlled or sustained release of the product which can be delivered via depot injection. In certain embodiments, hyaluronic acid may also be used to promote sustained duration in the circulation. In certain embodiments, implantable drug delivery devices may be used to introduce the desired at least one of an inhibitor of transforming growth factor-β (TGF-β), an inhibitor of connective tissue growth factor (CTGF), or an inhibitor of a SMAD protein.

Pharmaceutical compositions of the invention can be formulated for inhalation. In these embodiments, the at least one of an inhibitor of transforming growth factor-β (TGF-β), an inhibitor of connective tissue growth factor (CTGF), or an inhibitor of a SMAD protein are advantageously formulated as a dry, inhalable powder. In preferred embodiments, inhalation solutions of the at least one of an inhibitor of transforming growth factor-β (TGF-β), an inhibitor of connective tissue growth factor (CTGF), or an inhibitor of a SMAD protein may also be formulated with a propellant for aerosol delivery. In certain embodiments, solutions may be nebulized. Pulmonary administration and formulation methods therefore are further described in International Patent Application No. PCT/US94/001875, which is incorporated by reference and describes pulmonary delivery of chemically modified proteins.

It is also contemplated that formulations can be administered orally. Compositions of the at least one of an inhibitor of transforming growth factor-β (TGF-β), an inhibitor of connective tissue growth factor (CTGF), or an inhibitor of a SMAD protein hat are administered in this fashion can be formulated with or without carriers customarily used in the compounding of solid dosage forms such as tablets and capsules. In certain embodiments, a capsule may be designed to release the active portion of the formulation at the point in the gastrointestinal tract when bioavailability is maximized and pre-systemic degradation is minimized. Additional agents can be included to facilitate absorption of the at least one of an inhibitor of transforming growth factor-β (TGF-β), an inhibitor of connective tissue growth factor (CTGF), or an inhibitor of a SMAD protein. Diluents, flavorings, low melting point waxes, vegetable oils, lubricants, suspending agents, tablet disintegrating agents, and binders may also be employed.

A pharmaceutical composition of the invention is preferably provided to comprise an effective quantity of at least one of an inhibitor of transforming growth factor-β (TGF-β), an inhibitor of connective tissue growth factor (CTGF), or an inhibitor of a SMAD protein in a mixture with non-toxic excipients that are suitable for the manufacture of tablets. By dissolving the tablets in sterile water, or another appropriate vehicle, solutions may be prepared in unit-dose form. Suitable excipients include, but are not limited to, inert diluents, such as calcium carbonate, sodium carbonate or bicarbonate, lactose, or calcium phosphate; or binding agents, such as starch, gelatin, or acacia; or lubricating agents such as magnesium stearate, stearic acid, or talc.

Additional pharmaceutical compositions will be evident to those skilled in the art, including formulations involving at least one of an inhibitor of transforming growth factor-β (TGF-β), an inhibitor of connective tissue growth factor (CTGF), or an inhibitor of a SMAD protein in sustained- or controlled-delivery formulations. Techniques for formulating a variety of other sustained- or controlled-delivery means, such as liposome carriers, bio-erodible microparticles or porous beads and depot injections, are also known to those skilled in the art. See, for example, International Patent Application No. PCT/US93/00829, which is incorporated by reference and describes controlled release of porous polymeric microparticles for delivery of pharmaceutical compositions. Sustained-release preparations may include semipermeable polymer matrices in the form of shaped articles, e.g. films, or microcapsules. Sustained release matrices may include polyesters, hydrogels, polylactides (as disclosed in U.S. Pat. No. 3,773,919 and European Patent Application Publication No. EP 058481, each of which is incorporated by reference), copolymers of L-glutamic acid and gamma ethyl-L-glutamate (Sidman et al., 1983, Biopolymers 22:547-556), poly (2-hydroxyethyl-methacrylate) (Langer et al., 1981, J. Biomed. Mater. Res. 15:167-277 and Langer, 1982, Chem. Tech. 12:98-105), ethylene vinyl acetate (Langer et al., supra) or poly-D(-)-3-hydroxybutyric acid (European Patent Application Publication No. EP 133,988). Sustained release compositions may also include liposomes that can be prepared by any of several methods known in the art. See e.g., Eppstein et al., 1985, Proc. Natl. Acad. Sci. USA 82:3688-3692; European Patent Application Publication Nos. EP 036,676; EP 088,046 and EP 143,949, incorporated by reference.

Pharmaceutical compositions used for in vivo administration are typically provided as sterile preparations. Sterilization can be accomplished by filtration through sterile filtration membranes. When the composition is lyophilized, sterilization using this method may be conducted either prior to or following lyophilization and reconstitution. Compositions for parenteral administration can be stored in lyophilized form or in a solution. Parenteral compositions generally are placed into a container having a sterile access port, for example, an intravenous solution bag or vial having a stopper pierceable by a hypodermic injection needle.

Once the pharmaceutical composition has been formulated, it may be stored in sterile vials as a solution, suspension, gel, emulsion, solid, or as a dehydrated or lyophilized powder. Such formulations may be stored either in a ready-to-use form or in a form (e.g., lyophilized) that is reconstituted prior to administration.

The at least one of an inhibitor of transforming growth factor-β (TGF-(β), an inhibitor of connective tissue growth factor (CTGF), or an inhibitor of a SMAD protein useful in the methods of the invention can be admixed, encapsulated, conjugated or otherwise associated with other molecules, molecule structures or mixtures of compounds, for example, liposomes, receptor targeted molecules, oral, rectal, topical or other formulations, for assisting in uptake, distribution and/or absorption in a patient, using methods that are well known in the pharmaceutical arts.

The at least one of an inhibitor of transforming growth factor-β (TGF-(β), an inhibitor of connective tissue growth factor (CTGF), or an inhibitor of a SMAD protein of the invention may be administered orally, topically, parenterally, by inhalation or spray or rectally in dosage unit formulations containing conventional non-toxic pharmaceutically acceptable carriers, adjuvants and vehicles. The term parenteral as used herein includes percutaneous, subcutaneous, intravascular (e.g., intravenous), intramuscular, or intrathecal injection or infusion techniques and the like. In addition, there is provided a pharmaceutical formulation comprising at least one of an inhibitor of transforming growth factor-β (TGF-β), an inhibitor of connective tissue growth factor (CTGF), or an inhibitor of a SMAD protein and a pharmaceutically acceptable carrier. One or more compounds comprising at least one of an inhibitor of transforming growth factor-β (TGF-β), an inhibitor of connective tissue growth factor (CTGF), or an inhibitor of a SMAD protein I may be present in association with one or more non-toxic pharmaceutically acceptable carriers and/or diluents and/or adjuvants, and if desired other active ingredients. The pharmaceutical compositions containing at least one of an inhibitor of transforming growth factor-β (TGF-β), an inhibitor of connective tissue growth factor (CTGF), or an inhibitor of a SMAD protein may be in a form suitable for oral use, for example, as tablets, troches, lozenges, aqueous or oily suspensions, dispersible powders or granules, emulsion, hard or soft capsules, or syrups or elixirs.

Compositions intended for oral use may be prepared according to any method known to the art for the manufacture of pharmaceutical compositions and such compositions may contain one or more agents selected from the group consisting of sweetening agents, flavoring agents, coloring agents and preservative agents in order to provide pharmaceutically elegant and palatable preparations. Tablets contain the active ingredient in admixture with non-toxic pharmaceutically acceptable excipients that are suitable for the manufacture of tablets. These excipients may be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets may be uncoated or they may be coated by known techniques. In some cases such coatings may be prepared by known techniques to delay disintegration and absorption in the gastrointestinal tract and thereby provide a sustained action over a longer period. For example, a time delay material such as glyceryl monosterate or glyceryl distearate may be employed.

Formulations for oral use may also be presented as hard gelatin capsules, wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin or olive oil.

Formulations for oral use may also be presented as lozenges.

Aqueous suspensions contain at least one of an inhibitor of transforming growth factor-β (TGF-β), an inhibitor of connective tissue growth factor (CTGF), or an inhibitor of a SMAD protein in admixture with excipients suitable for the manufacture of aqueous suspensions. Such excipients are suspending agents, for example sodium carboxymethylcellulose, methylcellulose, hydropropyl-methylcellulose, sodium alginate, polyvinylpyrrolidone, gum tragacanth and gum acacia; dispersing or wetting agents may be a naturally-occurring phosphatide, for example, lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl p-hydroxybenzoate, one or more coloring agents, one or more flavoring agents, and one or more sweetening agents, such as sucrose or saccharin.

Oily suspensions may be formulated by suspending at least one of an inhibitor of transforming growth factor-β (TGF-β), an inhibitor of connective tissue growth factor (CTGF), or an inhibitor of a SMAD protein in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. Sweetening agents and flavoring agents may be added to provide palatable oral preparations. These compositions may be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents or suspending agents are exemplified by those already mentioned above. Additional excipients, for example sweetening, flavoring and coloring agents, may also be present.

Pharmaceutical compositions of the invention may also be in the form of oil-in-water emulsions. The oily phase may be a vegetable oil or a mineral oil or mixtures of these. Suitable emulsifying agents may be naturally-occurring gums, for example gum acacia or gum tragacanth, naturally-occurring phosphatides, for example soy bean, lecithin, and esters or partial esters derived from fatty acids and hexitol, anhydrides, for example sorbitan monooleate, and condensation products of the said partial esters with ethylene oxide, for example polyoxyethylene sorbitan monooleate. The emulsions may also contain sweetening and flavoring agents.

Syrups and elixirs may be formulated with sweetening agents, for example glycerol, propylene glycol, sorbitol, glucose or sucrose. Such formulations may also contain a demulcent, a preservative, flavoring and/or coloring agents. The pharmaceutical compositions may be in the form of a sterile injectable aqueous or oleaginous suspension. This suspension may be formulated according to the known art using those suitable dispersing or wetting agents and suspending agents that have been mentioned above. The sterile injectable preparation may also be a sterile injectable solution or suspension in a non-toxic parentally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono-or diglycerides. In addition, fatty acids such as oleic acid find use in the preparation of injectables.

The at least one of an inhibitor of transforming growth factor-β (TGF-β), an inhibitor of connective tissue growth factor (CTGF), or an inhibitor of a SMAD protein of the invention may also be administered in the form of suppositories, e.g., for rectal administration of the drug. These compositions can be prepared by mixing the drug with a suitable non-irritating excipient that is solid at ordinary temperatures but liquid at the rectal temperature and will therefore melt in the rectum to release the drug. Such materials include cocoa butter and polyethylene glycols.

The at least one of an inhibitor of transforming growth factor-β (TGF-β), an inhibitor of connective tissue growth factor (CTGF), or an inhibitor of a SMAD protein of the invention may be administered parenterally in a sterile medium. The drug, depending on the vehicle and concentration used, can either be suspended or dissolved in the vehicle. Advantageously, adjuvants such as local anesthetics, preservatives and buffering agents can be dissolved in the vehicle.

Alternatively, the at least one of an inhibitor of transforming growth factor-β (TGF-β), an inhibitor of connective tissue growth factor (CTGF), or an inhibitor of a SMAD protein may be formulated in a cream with an oil-in-water cream base. If desired, the aqueous phase of the cream base may include, for example at least 30% w/w of a polyhydric alcohol such as propylene glycol, butane-1,3-diol, mannitol, sorbitol, glycerol, polyethylene glycol and mixtures thereof. The topical formulation may desirably include a compound that enhances absorption or penetration of the at least one of an inhibitor of transforming growth factor-β (TGF-β), an inhibitor of connective tissue growth factor (CTGF), or an inhibitor of a SMAD protein through the skin or other affected areas. Examples of such dermal penetration enhancers include dimethylsulfoxide and related analogs.

The compounds of this invention can also be administered by a transdermal device. Preferably topical administration will be accomplished using a patch either of the reservoir and porous membrane type or of a solid matrix variety. In either case, the at least one of an inhibitor of transforming growth factor-β (TGF-β), an inhibitor of connective tissue growth factor (CTGF), or an inhibitor of a SMAD protein is delivered continuously from the reservoir or microcapsules through a membrane into the active agent permeable adhesive, which is in contact with the skin or mucosa of the recipient. If the active agent is absorbed through the skin, a controlled and predetermined flow of the at least one of an inhibitor of transforming growth factor-β (TGF-β), an inhibitor of connective tissue growth factor (CTGF), or an inhibitor of a SMAD protein is administered to the recipient. In the case of microcapsules, the encapsulating agent may also function as the membrane. The transdermal patch may include the at least one of an inhibitor of transforming growth factor-β (TGF-β), an inhibitor of connective tissue growth factor (CTGF), or an inhibitor of a SMAD protein in a suitable solvent system with an adhesive system, such as an acrylic emulsion, and a polyester patch.

The oily phase of the emulsions of this invention may be constituted from known ingredients in a known manner. While the phase may comprise merely an emulsifier, it may comprise a mixture of at least one emulsifier with a fat or an oil or with both a fat and an oil. Preferably, a hydrophilic emulsifier is included together with a lipophilic emulsifier that acts as a stabilizer. It is also preferred to include both an oil and a fat. Together, the emulsifier(s) with or without stabilizer(s) make-up the so-called emulsifying wax, and the wax together with the oil and fat make up the so-called emulsifying ointment base which forms the oily dispersed phase of the cream formulations. Emulsifiers and emulsion stabilizers suitable for use in the formulation of the present invention include Tween 60, Span 80, cetostearyl alcohol, myristyl alcohol, glyceryl monostearate, and sodium lauryl sulfate, among others.

The choice of suitable oils or fats for the formulation is based on achieving the desired cosmetic properties, since the solubility of the active compound in most oils likely to be used in pharmaceutical emulsion formulations is very low. Thus, the cream should preferably be a non-greasy, non-staining and washable product with suitable consistency to avoid leakage from tubes or other containers. Straight or branched chain, mono- or dibasic alkyl esters such as di-isoadipate, isocetyl stearate, propylene glycol diester of coconut fatty acids, isopropyl myristate, decyl oleate, isopropyl palmitate, butyl stearate, 2-ethylhexyl palmitate or a blend of branched chain esters may be used. These may be used alone or in combination depending on the properties required. Alternatively, high melting point lipids such as white soft paraffin and/or liquid paraffin or other mineral oils can be used.

For therapeutic purposes, the at least one of an inhibitor of transforming growth factor-β (TGF-β), an inhibitor of connective tissue growth factor (CTGF), or an inhibitor of a SMAD protein of this invention are ordinarily combined with one or more adjuvants appropriate to the indicated route of administration. If administered by mouth, the compounds may be admixed with lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia gum, sodium alginate, polyvinylpyrrolidone, and/or polyvinyl alcohol, and then tableted or encapsulated for convenient administration. Such capsules or tablets may contain a controlled-release formulation as may be provided in a dispersion of active compound in hydroxypropylmethyl cellulose. Formulations for parenteral administration may be in the form of aqueous or non-aqueous isotonic sterile injection solutions or suspensions. These solutions and suspensions may be prepared from sterile powders or granules having one or more of the carriers or diluents mentioned for use in the formulations for oral administration. The compounds may be dissolved in water, polyethylene glycol, propylene glycol, ethanol, corn oil, cottonseed oil, peanut oil, sesame oil, benzyl alcohol, sodium chloride, and/or various buffers. Other adjuvants and modes of administration are well and widely known in the pharmaceutical art.

The amount of at least one of an inhibitor of transforming growth factor-β (TGF-β), an inhibitor of connective tissue growth factor (CTGF), or an inhibitor of a SMAD protein that may be combined with the carrier materials to produce a single dosage form will vary depending upon the host treated and the particular mode of administration. Dosage unit forms will generally contain at least one of an inhibitor of transforming growth factor-β (TGF-β), an inhibitor of connective tissue growth factor (CTGF), or an inhibitor of a SMAD protein.

It will be understood, however, that the specific dose level for any particular patient will depend upon a variety of factors including the activity of the at least one of an inhibitor of transforming growth factor-β (TGF-β), an inhibitor of connective tissue growth factor (CTGF), or an inhibitor of a SMAD protein employed, the age, body weight, general health, sex, diet, time of administration, route of administration, and rate of excretion, drug combination and the severity of the particular disease undergoing therapy.

For administration to non-human animals, the composition may also be added to the animal feed or drinking water. It may be convenient to formulate the animal feed and drinking water compositions so that the animal takes in a therapeutically appropriate quantity of the at least one of an inhibitor of transforming growth factor-β (TGF-β), an inhibitor of connective tissue growth factor (CTGF), or an inhibitor of a SMAD protein along with its diet. It may also be convenient to present the composition as a premix for addition to the feed or drinking water.

Dosing frequency will depend upon the pharmacokinetic parameters of the particular at least one of an inhibitor of transforming growth factor-β (TGF-β), an inhibitor of connective tissue growth factor (CTGF), or an inhibitor of a SMAD protein used in the formulation. Typically, a clinician administers the composition until a dosage is reached that achieves the desired effect. The composition may therefore be administered as a single dose, or as two or more doses (which may or may not contain the same amount of the desired molecule) over time, or as a continuous infusion via an implantation device or catheter. Further refinement of the appropriate dosage is routinely made by those of ordinary skill in the art and is within the ambit of tasks routinely performed by them. Appropriate dosages may be ascertained through use of appropriate dose-response data. In certain embodiments, the at least one of an inhibitor of transforming growth factor-β (TGF-β), an inhibitor of connective tissue growth factor (CTGF), or an inhibitor of a SMAD protein can be administered to patients throughout an extended time period.

Pharmaceutical compositions of the at least one of an inhibitor of transforming growth factor-β (TGF-β), an inhibitor of connective tissue growth factor (CTGF), or an inhibitor of a SMAD protein of the invention can be administered alone or in combination with other therapeutic agents, in particular, in combination with other anti-fibrotic agents.

The invention also provides kits for producing a single-dose administration unit. The kits of the invention may each contain both a first container having dried embodiments of the at least one of an inhibitor of transforming growth factor-β (TGF-β), an inhibitor of connective tissue growth factor (CTGF), or an inhibitor of a SMAD protein and a second container having an aqueous or other appropriate solution for formulating the final dosage form. In certain embodiments of this invention, kits containing single and multi-chambered pre-filled syringes (e.g., liquid syringes and lyosyringes) are provided.

Unless otherwise required by context, singular terms used herein shall include pluralities and plural terms shall include the singular.

### Examples

The following examples, including the experiments conducted and results achieved are provided for illustrative purposes only and are not to be construed as limiting the invention.

### Example 1

### Senescence-Prone Mice Showed Accelerated Aging at 6 Months of Age.

Genetically-selected senescence-prone mice were used to compare diastolic function with normal control animals. Mice bearing the accelerated senescence phenotype were derived from AKR/J mice by continuous sister-brother mating selecting for a tendency toward either accelerated or normal senescence; breeders were retrospectively chosen based on the degree of senescence at eight months as determined by life span and clinical signs of aging (Takeda et al., 1981, A new murine model of accelerated senescence. Mech Ageing Dev 17: 183-194). The SAM model is comprised of the senescence-prone (SAMP) and control senescence-resistant (SAMR) strains. As understood in the art, the “accelerated senescence” phenotype refers to the tendency of SAMP mice to experience a rapid progression of senescence after reaching maturity and to have a life span shortened by about 40% when compared to SAMR mice (Takeda, 1999, Senescence-accelerated mouse (SAM): a biogerontological resource in aging research. Neurobiol Aging 20: 105-110). The median life span of SAMR1 mice has been reported to be between 12 and 21 months of age, and the median life span of SAMP8 mice has been reported as 10 to 17 months of age (Flood & Morley, 1998, Learning and memory in the SAMP8 mouse. Neurosci Biobehav Rev 22: 1-20)(although some variability exists with respect to life span of these mice). Pathological changes detected at autopsy in these mice after their natural death include pneumonia, abscess, colitis, amyloidosis, contracted kidney, and neoplasms among others, while the most frequent causes of death in SAMP mice are lymphoid neoplasms and contracted kidney (Takeda et al., 1997, Pathobiology of the senescence-accelerated mouse (SAM). Exp Gerontol 32: 117-127). SAMR1 and SAMP8 strains were used to assess age-associated diastolic dysfunction since these are the best-studied strains with respect to cardiovascular disease and oxidative stress.

SAMR1 and SAMP8 mice were purchased from Harlan (Indianapolis, Ind.). All experiments were carried out using 3-month-old and 6-month-old male mice and conformed with U.S. National Institutes of Health (Guide for the Care and Use of Laboratory Animals) and institutional animal care guidelines. Initially, SAMP8 mice were tested to assess the presence of the accelerated senescence phenotype. In these experiments, levels of p19^{ARF} (also known as ARF) were measured using quantitative real-time PCR; this protein is a tumor suppressor encoded by the INK4a/ARF locus and regulates the p53 pathway by stabilizing p53. In these experiments, total RNA was isolated from left ventricular heart tissue homogenates and was reverse transcribed using the SuperScript II kit (Invitrogen, San Diego, Calif.). cDNA was purified and then amplified using gene-specific primers and a LightCycler real-time thermocycler (Roche Diagnostics Corp, Indianapolis, Ind.). Transcripts were detected using SYBR Green I (Molecular Probes, Inc) and were normalized to 18S mRNA.

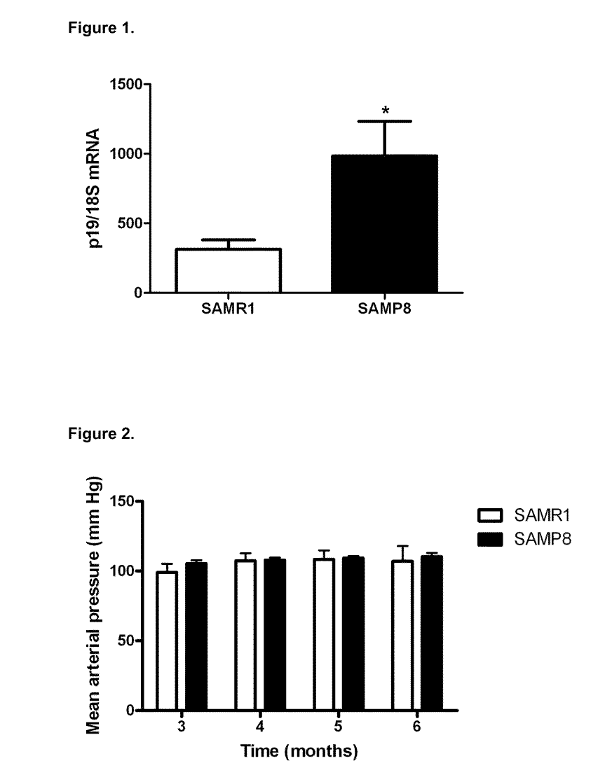

The results of these experiments showed that p19^{ARF} levels were increased in 6 month old SAMP8 mice compared to SAMR1 controls as measured by quantitative real-time PCR (qRT-PCR) (FIG. 1). This finding was consistent with aging and accelerated senescence in SAMP8 mice, since senescence requires activation of the p53 pathway (Sharpless, 2004, Ink4a/Arf links senescence and aging. Exp Gerontol 39: 1751-1759).

### Example 2

### Senescence-Prone Mice Showed Age-Related Diastolic Dysfunction

These mice where then tested for diastolic dysfunction using echocardiography. Different groups of mice were used for experiments studies carried out at 3, 6, and 12 months of age. Briefly, mice were anesthetized with 4% isoflurane, hair was removed from the thorax, and they were maintained under light anesthesia (1-1.5% isoflurane) at approximately 37° C. and demonstrated a physiological heart rate >500 bpm during the procedure. Two-dimensional and M-mode transthoracic echocardiography modalities were used to assess heart wall motion, chamber dimensions, and wall thickness and to calculate fractional shortening. Measurements were made using a VisualSonics® Vevo 770TM in-vivo micro-imaging system equipped with a RMV-707B cardiovascular scanhead (Toronto, ON). Pulsed-wave Doppler echocardiography was used to measure early (E) and late (A) blood flow velocities through the mitral valve. Tissue Doppler imaging was used to measure the early (E′) and late (A′) velocity of the mitral annulus. For each measured data point, at least 3 beats were averaged per measurement, at least 3 measurements were taken per animal, and beats were taken at end expiration. The experimental protocol used was also as set forth in Silberman et al. (2010, Uncoupled cardiac nitric oxide synthase mediates diastolic dysfunction. Circulation 121: 519-528, incorporated by reference).

Results of these experiments are shown in Table 1. At 6 months of age, SAMP8 mice displayed echocardiographic evidence of diastolic dysfunction compared to SAMR1 controls. Using conventional pulsed-wave Doppler echocardiography, the ratio of early to late mitral inflow velocity (E/A) was reduced in SAMP8 mice compared to SAMR1 controls (1.2±0.03 vs. 1.3±0.03. p<0.05). Tissue Doppler imaging was used to measure the mitral valve annulus velocity. In SAMP8 mice, the tissue mitral annulus early longitudinal velocity (E′) was reduced compared to SAMR1 controls (21.1±0.8 vs. 25.7±0.9 mm/s, p<0.05). Likewise, the ratio of early to late tissue mitral annulus velocities (E′/A′) was reduced in SAMP8 mice (0.8±0.03 vs. 1.1±0.02, p<0.05) (Table 1). The dependency of the phenotype upon age was confirmed with echocardiographic studies performed at three months of age that showed no differences in diastolic function (Table 1). Furthermore, experiments using 12 month old SAMR1 and SAMP8 mice confirmed that diastolic dysfunction in the model was age-related. In 12 month old SAMP8 mice, the E′/A′ ratio was reduced compared to SAMR1 controls (0.8±0.03 vs. 1.0±0.04, p<0.05) (Table 2). In addition, when diastolic function was compared between both types of mice at 6 and 12 months of age, diastolic function apparently further deteriorated in SAMP8 mice and even began to deteriorate in SAMR1 controls by 12 months of age.

TABLE 1
Echocardiographic comparison of SAMR1 and SAMP8 mice at 3 and 6 months of age.

| | SAMR1 at | SAMP8 at | SAMR1 at | SAMP8 at | |
|---|---|---|---|---|---|
| | 3 months | 3 months | 6 months | 6 months | |
| | (n = 7) | (n = 7) | (n = 8) | (n = 8) | p value |
| Body weight (g) | 29.0 ± 0.3 | 30.7 ± 0.5 | §39.1 ± 1.1 | *§42.3 ± 1.0 | <0.05 |
| LV weight (mg) | 82.0 ± 2.2 | *90.5 ± 2.3 | §110.4 ± 1.9 | *§120.0 ± 2.2 | <0.05 |
| LV/body weight | 2.8 ± 0.05 | 2.9 ± 0.4 | 2.8 ± 0.07 | 2.9 ± 0.08 | NS |
| LV dimensions | | | | | |
| LVID; s (mm) | 2.9 ± 0.07 | 2.7 ± 0.1 | 2.6 ± 0.07 | 2.6 ± 0.07 | NS |
| LVID; d (mm) | 4.0 ± 0.05 | 4.0 ± 0.1 | 4.0 ± 0.06 | 4.0 ± 0.08 | NS |
| LV vol; s (μL) | 32.3 ± 1.8 | 28.1 ± 2.8 | 25.1 ± 1.7 | 24.8 ± 1.7 | NS |
| LV vol; d (μL) | 71.6 ± 2.2 | 70.9 ± 4.7 | 69.7 ± 2.3 | 70.5 ± 3.6 | NS |
| Diastolic | | | | | |
| E/A | 1.4 ± 0.03 | 1.4 ± 0.04 | 1.3 ± 0.03 | *§1.2 ± 0.03 | <0.05 |
| E′ (mm/s) | 28.1 ± 1.03 | 30.8 ± 2.0 | 25.7 ± 0.9 | §21.1 ± 0.8 | <0.05 |
| A′ (mm/s) | 20.7 ± 0.9 | 20.8 ± 1.7 | 23.3 ± 0.8 | §25.8 ± 1.1 | <0.05 |
| E′/A′ | 1.4 ± 0.03 | 1.4 ± 0.04 | §1.1 ± 0.02 | *§0.8 ± 0.03 | *§<0.05 |

LV, left ventricle; SV, stroke volume; EF, percent ejection fraction; FS, percent fractional shortening; E/A, ratio of early to late diastolic filling measured by pulsed-wave Doppler; E′, mitral annulus velocity during early diastole measured by tissue Doppler imaging (TDI); A′, mitral annulus velocity during late diastole measured by TDI; E′/A′, ratio of mitral annulus velocities during early and late diastolic filling measured by TDI. Values were compared between SAMR1 and SAMP8 groups at 3 and 6 months of age.
*p value is significant when comparison is made between SAMR1 and SAMP8 mice of the same age, and §p value is significant when comparison is made between the same type of mice at 3 and 6 months of age.

TABLE 2
Echocardiographic comparison of SAMR1
and SAMP8 mice at 12 months of age.

| | SAMR1 at | SAMP8 at | |
|---|---|---|---|
| | 12 months | 12 months | |
| | (n = 6) | (n = 6) | p value |
| Body weight (g) | 50.5 ± 1.4 | 44.7 ± 1.8 | <0.05 |
| LV weight (mg) | 146.5 ± 2.6 | 144.9 ± 2.8 | NS |
| LV/body weight | 2.9 ± 0.06 | 3.3 ± 0.17 | <0.05 |
| LV dimensions | | | |
| LVID; s (mm) | 2.8 ± 0.14 | 2.9 ± 0.12 | NS |
| LVID; d (mm) | 4.2 ± 0.09 | 4.3 ± 0.09 | NS |
| LV vol; s (μL) | 32.6 ± 3.6 | 34.0 ± 3.5 | NS |
| LV vol; d (μL) | 80.7 ± 3.8 | 83.8 ± 4.1 | NS |
| Diastolic | | | |
| E/A | 1.1 ± 0.04 | 1.1 ± 0.04 | NS |
| E′ (mm/s) | 22.9 ± 0.7 | 22.8 ± 0.9 | NS |
| A′ (mm/s) | 22.9 ± 0.9 | 25.3 ± 1.1 | NS |
| E′/A′ | 1.0 ± 0.04 | 0.8 ± 0.03 | <0.05 |

HR, heart rate; LVESV, LV end-systolic volume; LVEDV, LV end-diastolic volume; LVESP, LV end-systolic pressure; LVEDP, LV end-diastolic pressure, dP/dtₘₐₓ, maximal slope of left ventricular pressure rise during systole; dP/dtₘᵢₙ, maximal slope of left ventricular pressure decline during diastole; dP/dt_{EDV}, dP/dt divided by end-diastolic volume; Ea, arterial elastance; Ea/Es, ventricular-vascular coupling ratio; Tau-Glantz, time constant of pressure decay by the Glantz method; Tau-Weiss, time constant of pressure decay by the Weiss method; Tau_{1/2}, half-time of the time constant of pressure decay; EDPVR, end-diastolic pressure-volume relationship; ESPVR, end-systolic pressure-volume relationship; PRSW, preload recruitable stroke work.

These echocardiographic observations were confirmed using invasive hemodynamics. In these experiments, mice were anesthetized with 1-2% isoflurane. An initial intraperitoneal bolus injection of 0.3 mL normal saline was given, and the body temperature was maintained at 37-38° C. during the procedure. After a single dose of pancuronium (0.12 mg/kg IV) through left jugular vein, mechanical ventilation (MA1 55-7059, Harvard Apparatus, Holliston, Mass., USA) was started via tracheoectomy with a rate of 118-133 breaths per min and a tidal volume of 0.19-0.28 mL. A pressure-volume catheter (SPR-839, Millar Instruments, Houston, Tex., USA) was inserted into the right common carotid artery and advanced into the left ventricle. Inferior vena cava occlusion was performed via a midline abdominal incision. Volume and parallel conductance calibration were performed as previously described Yang et al., 2001, Validation of conductance catheter system for quantification of murine pressure-volume loops. J Invest Surg 14: 341-355, incorporated by reference). The group of mice used for invasive hemodynamic studies was separate from the group used for echocardiography; however, all mice were 6 months old at the time of study. The experimental protocol used was for invasive hemodynamic studies were as described previously using a closed-chest procedure (see Silberman et al., Id.).

These studies showed that SAMP8 mice displayed hemodynamic evidence of diastolic dysfunction compared to SAMR1 controls. Compared to SAMR1 mice at 6 months of age, SAMP8 mice had an increased end-diastolic pressure (5.6±0.9 vs. 3.4±0.3 mmHg, p<0.05). Additionally, transient occlusion of the inferior vena cava was used to generate a family of pressure-volume loops at varying volumes. The left ventricular end-diastolic pressure-volume relationship (EDPVR) was determined and represented as the slope of the best-fitting line connecting the end-diastolic pressure-volume points, corresponding to the passive physical properties of the left ventricle Burkhoff et al. (2005, Assessment of systolic and diastolic ventricular properties via pressure-volume analysis: a guide for clinical, translational, and basic researchers. Am J Physiol Heart Circ Physiol 289: H501-512, incorporated by reference.) Consistent with the data set forth above indicating diastolic dysfunction in SAMP8 mice, the slope of the EDPVR was found to be increased in SAMP8 mice compared to SAMR1 controls (0.8±0.1 vs. 0.5±0.05 mmHg/μL, p<0.05) (Table 2). No differences in dP/dtₘᵢₙ, the maximal slope of ventricular pressure decline during diastole or in Tau-Glantz, Tau-Weiss, or Tau_{1/2},. (three different ways of measuring the time constant of pressure decay) were observed, however, suggesting that the pressure-volume relation, and hence passive filling, but not active relaxation was affected in these mice. Arterial elastance (Ea) and the ventricular-vascular coupling ratio (Ea/Es) were also measured to determine whether abnormal ventricular-vascular coupling could account for the changes in cardiac function we observed. There were no differences between SAMP8 and SAMR1 mice in Ea (8.8±1.1 vs. 7.0±0.5) or Ea/Es (1.2±0.2 vs. 1.3±0.2), respectively, suggesting changes are not the result of altered ventricular-vascular coupling.

In addition, conventional M-mode echocardiography was used to measure cardiac dimensions. There were no differences in left ventricular dimensions between SAMR1 and SAMP8 mice during either systole or diastole. Furthermore, the stroke volume (44.5±1.0 vs. 45.7±2.2 μL), the ejection fraction (64.3±1.5 vs. 65.0±1.2.%), and the percent fractional shortening (34.7±1.1 vs. 35.3±0.8%) were unchanged between SAMP8 and SAMR1 mice, respectively, suggesting that changes in diastolic function could not be explained by changes in systolic function (Table 3).

TABLE 3
Invasive hemodynamic comparison of SAMR1
and SAMP8 mice at 6 months of age.

| | SAMR1 | SAMP8 | |
|---|---|---|---|
| | (n = 11) | (n = 8) | p value |
| Baseline HR (bpm) | 603.1 ± 12.05 | 583.4 ± 11.41 | NS |
| LVESP (mmHg) | 85.82 ± 3.37 | 79.50 ± 4.00 | NS |
| LVEDP (mmHg) | 3.41 ± 0.28 | 5.59 ± 0.93 | <0.05 |
| dP/dtₘₐₓ (mmHg/sec) | 8093 ± 721.2 | 7534 ± 787.7 | NS |
| dP/dtₘᵢₙ (mmHg/sec) | −9138 ± 831.8 | −9089 ± 1055 | NS |
| dP/dt_{EDV} (mmHg/sec) | 461.7 ± 68.00 | 624.5 ± 102.5 | NS |
| Ea (mmHg/μL) | 6.95 ± 0.45 | 8.79 ± 1.10 | NS |
| Ea/Es | 1.30 ± 0.16 | 1.23 ± 0.18 | NS |
| Tau-Glantz (ms) | 8.50 ± 0.59 | 8.66 ± 0.66 | NS |
| Tau-Weiss (ms) | 5.09 ± 0.26 | 5.73 ± 0.43 | NS |
| t_{1/2} | 4.03 ± 0.18 | 4.13 ± 0.18 | NS |
| EDPVR (mmHg/μL) | 0.49 ± 0.05 | 0.79 ± 0.14 | <0.05 |
| ESPVR (mmHg/μL) | 5.90 ± 0.59 | 7.87 ± 1.00 | NS |
| PRSW | 72.11 ± 8.39 | 65.40 ± 11.47 | NS |

SV, stroke volume; EF, percent ejection fraction; FS, percent fractional shortening; E/A, ratio of early to late diastolic filling measured by pulsed-wave Doppler; E′, mitral annulus velocity during early diastole measured by tissue Doppler imaging (TDI); A′, mitral annulus velocity during late diastole measured by TDI; E′/A′, ratio of mitral annulus velocities during early and late diastolic filling measured by TDI. Values were compared between SAMR1 and SAMP8 groups at 6 months of age.

### Example 3

### Diastolic Dysfunction in Senescence-Prone Mice Not Related to Hypertension or Cardiomyocyte Dysfunction

Since hypertension is an established risk factor for the development of diastolic dysfunction (Zile & Brutsaert, 2002, New concepts in diastolic dysfunction and diastolic heart failure: Part I: diagnosis, prognosis, and measurements of diastolic function. Circulation 105: 1387-1393), mean arterial pressure was monitored by telemetry in SAMR1 and SAMP8 mice from 3 through 6 months of age. Blood pressure was measured as described in Kleinhenz et al. (2009, Disruption of endothelial peroxisome proliferator-activated receptor-gamma reduces vascular nitric oxide production. Am J Physiol Heart Circ Physiol 297: H1647-1654, incorporated by reference). Briefly, anesthesia was induced using 4% isoflurane and maintained at 1-1.5% isoflurane. A 2-cm ventral incision was made from the chin to the sternum, and the carotid artery was isolated by blunt dissection. A 25-guage bent needle was used to cannulate the artery with a sterile TA11PA-C10 transmitter (Data Sciences International, St. Paul, Minn.). The catheter connected to the transducer was advanced into the thoracic aorta and held in place with sutures, and the transmitter was positioned along the right flank, close to the hindlimb. Mice were allowed to recover for 1 week prior to initiation of monitoring. Blood pressure measurements were telemetrically collected for 10 s each minute during a 24-hour period at the baseline time point of 3 months of age, and then weekly up to 6 months of age. The group of mice used for telemetry was separate from the groups used for echocardiography and invasive hemodynamics.

The experimental results showed no differences in mean arterial pressure between the two groups of mice at any time point, and there was no change in pressure over the three month course of measurement (FIG. 2). Therefore, changes in diastolic function observed were independent of blood pressure changes.

Since diastolic dysfunction could be due to impairment in the relaxation of cardiac myocytes, sarcomere length shortening and relengthening were examined. Relaxation was measured in freshly isolated ventricular cardiomyocytes. The mechanical properties of the cardiomyocytes were assessed using an lonOptix Myocam System (lonoptix Inc., Milton, Mass.). Unloaded cardiomyocytes were placed on a glass slide and allowed to adhere for 10 min. Cardiomyocytes were then imaged with an inverted microscope and perfused with a normal Tyrode's solution containing 1.2 mmol/L CaCl₂ at 37° C. by temperature controller and heater (mTC-II, lonoptix Inc., Milton, Mass.). Cardiomyocytes were paced at 1.0 Hz for 4 ms duration, and sarcomere shortening and relengthening were assessed using the following indices: peak fractional shortening (FS), time to 90% peak shortening, and Tau, the relaxation time constant (a₀+a₁e^{tτ}, t=time). Cardiomyocytes (47-59) in 5-6 different mice were used to measure sarcomere shortening. A separate group of 6-month-old male mice was used for sacrcomere length shortening measurements.

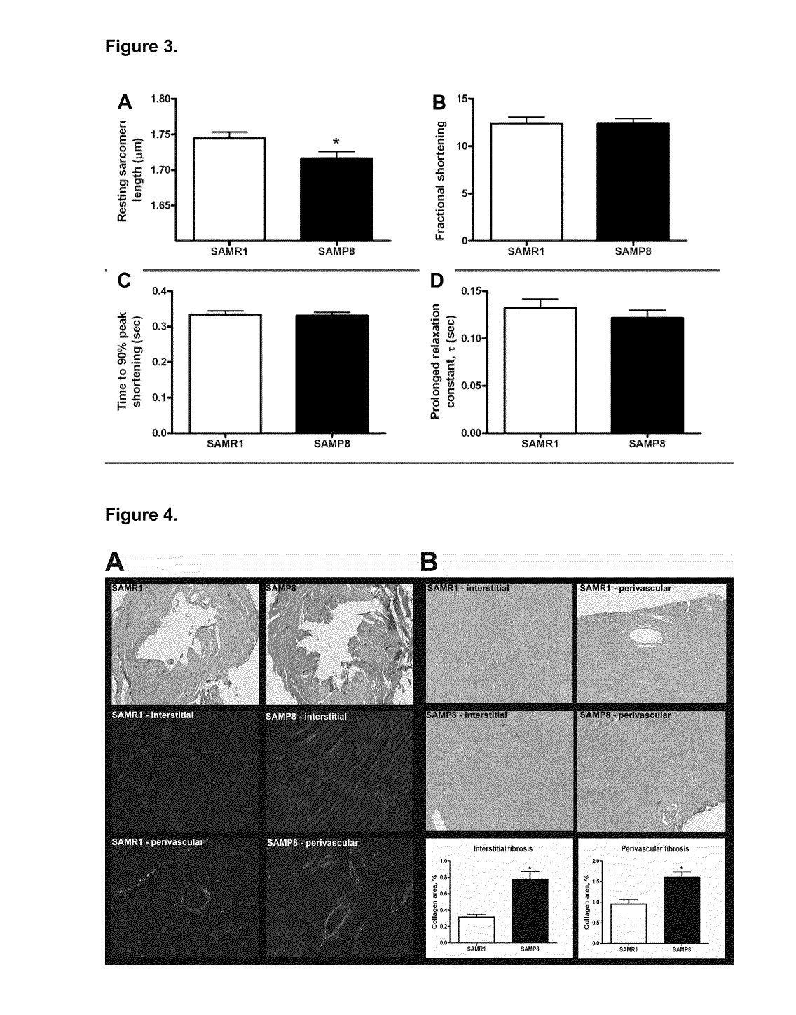

The results of these experiments are shown in FIGS. 3B, 3C and 3D. Baseline sarcomere length of cardiac myocytes determined as set forth above was modestly shorter in SAMP8 mice compared to SAMR1 mice (1.72±0.01 μm vs. 1.78±0.01 μm, p<0.05) (FIG. 3A). Nevertheless, there were no significant differences in fractional shortening (12.42±0.67% vs. 12.43±0.48%), time to 90% peak shortening (0.33±0.01 sec vs. 0.33±0.01 sec), and Tau, the relaxation time constant (0.13±0.01 vs. 0.12±0.01) between SAMR1 and SAMP8 mice, respectively (FIG. 3B, 3C and 3D). These result suggested that diastolic dysfunction observed in this model was not the result of changes in cardiomyocyte function.

Finally, since these mice were known to develop other pathologies in addition to those related to cardiac function and because cardiac and renal function are often interrelated, the metabolic profiles of SAMP8 and SAMR1 mice were determined at six months of age. Plasma blood urea nitrate (BUN) was 15.9±0.5 in six-month-old SAMR1 mice and was only mildly elevated to 17.8±0.4 in SAMP8 mice. Plasma creatinine was unchanged in SAMR1 versus SAMP8 mice (0.21±0.01 vs. 0.20±0.0). Finally, the diastolic dysfunction observed did not progress fully to diastolic heart failure; there was no observed difference in lung weights between SAMR1 and SAMP8 mice at 6 months of age.

These results eliminated various explanations for the senescence-related diastolic dysfunction observed in these mice.

### Example 4

### Senescence-Prone Mice Demonstrated Increased Cardiac Fibrosis.

In view of the results showing that diastolic dysfunction in SAMP8 mice was not secondary to hypertension, other causes and mechanisms of the age-associated diastolic dysfunction in these mice were investigated. These included causes possibly related to stiffening of the left ventricle and diminished distensibility of the heart muscle observed in a population of human patients with diastolic dysfunction (Borlaug & Kass, 2005, Mechanisms of diastolic dysfunction in heart failure. Trends Cardiovasc Med 16: 273-279). Fibrotic responses in SAM mice were thus investigated. In these experiments, the content of myocardial collagen (an important component of fibrotic tissue) was examined using two different histological methods: picrosirius staining and Masson's trichrome staining (Catalog No. AR173, Dako North America, Inc, Carpinteria, Calif.; see also Masson, 1929, Some histological methods. Trichrome stainings and their preliminary technique. J. Techn. Methods 12: 75-90, incorporated by reference). In a first set of experiments, hearts from SAMR1 and SAMP8 mice were fixed in 10% buffered formalin for 24 hours, embedded in paraffin, and 5 μm transverse sections were cut using a microtome. Tissue sections were de-waxed, rehydrated, and then stained with picrosirius red. For this staining, sections were stained in a 0.1% solution of sirius red in a saturated aqueous solution of picric acid for 1 hour then washed in acidified water, dehydrated with graded alcohols, and mounted on slides. Both bright-field and polarized light microscopy were used to image and photograph the slides. Slides were imaged using a using a Zeiss microscope and AxioVision 4.5 software. For slides stained with Masson's trichrome, ImagePro 6.2 software was used to calculate the percent area of collagen content.

When transverse myocardial tissue sections were stained with sirius red, greater collagen accumulation in whole hearts of SAMP8 mice was evident due to increased red staining of the tissue using brightfield microscopy (FIG. 4A, upper panels). Tissue sections were also imaged using polarized light, where large collagen fibers appeared yellow or orange, and thinner fibers appeared green (Junqueira et al., 1979, Picrosirius staining plus polarization microscopy, a specific method for collagen detection in tissue sections. Histochem J 11: 447-455). In both interstitial and perivascular regions of the myocardium, SAMP8 mice showed increased collagen deposition compared to SAMR1 controls. The accumulation of large collagen fibers was particularly markedly increased in the perivascular regions of hearts from SAMP8 mice (FIG. 4B, lower panels). To confirm and quantify these findings, Masson's trichrome staining was used. Again, greater collagen accumulation was observed in the interstitial and perivascular regions of the myocardium in SAMP8 mice. In the interstitial and perivascular tissue, the percentage of tissue comprised of collagen was greater in SAMP8 mice compared to SAMR1 controls (0.8±0.1 vs. 0.3±0.04, p<0.05) and (1.6±0.1 vs. 1.0±0.1, p<0.05), respectively (FIG. 4B).

To further examine which collagens and other associated proteins might be increased in the myocardium of senescence-accelerated mice, quantitative real-time PCR was used to measure myocardial gene expression of collagen 1A1, collagen 3A, and fibronectin. These experiments were performed as set forth in Example 1 above, using gene-specific primers as follows:

(SEQ ID NO: 1)
collagen 1A1 (+1) CTAAGGGTCCCCAATGGTGAGAC
(SEQ ID NO: 2)
collagen 1A1 (−2) GGGGGTTGGGACAGTCCAGTTCTTC
(SEQ ID NO: 3)
collagen 3A (+1) CCCAACCCAGAGATCCCATTTGGAG
(SED ID NO: 4)
collagen 3A (−2) GGCCACCAGTTGGACATGATTCACA
(SEQ ID NO: 5)
fibronectin 1 (+1) CTCAACCTCCCTGAAACGGCCAACT
(SEQ ID NO: 6)
fibronectin 1 (−2) TCTTGGGGTGCCAGTGGTCTCTTGT.

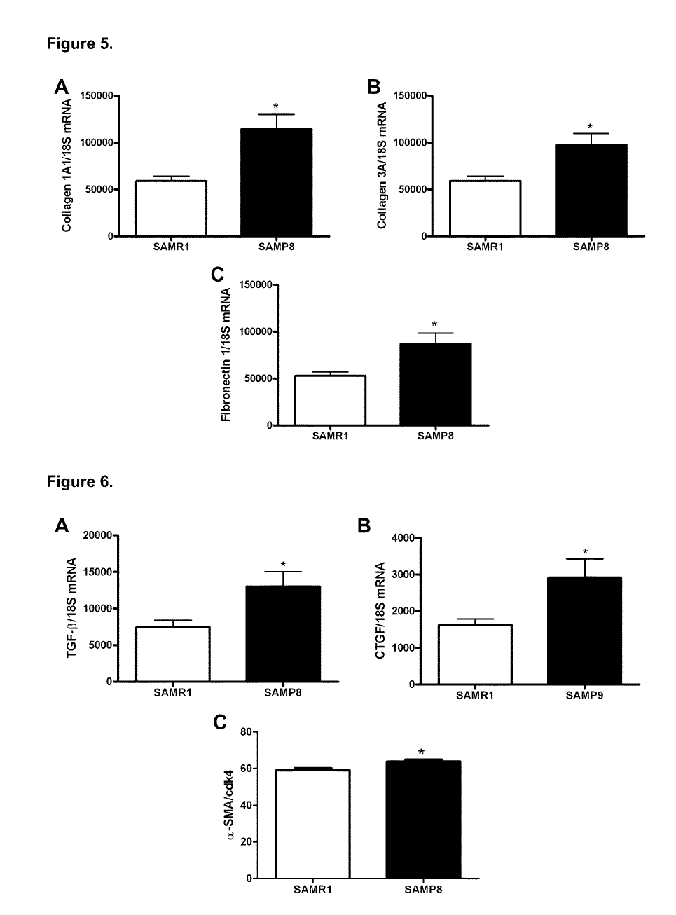

In these experiments it was found that collagen 1A1, which is the main component of scar tissue, and collagen 3A, commonly associated with collagen 1A1, were increased in SAMP8 mice compared to SAMR1 controls (FIGS. 5A and 5B). Expression of fibronectin 1 (an extracellular matrix protein that can bind to collagen) was also increased in SAMP8 mice compared with SAMR1 control mice (FIG. 5C).

These results indicated that senescence-related diastolic dysfunction was associated with a fibrotic response.

### Example 5

### Cardiac Fibrosis Observed in Senescence-Accelerated Mice was Associated with Increased Expression of Pro-Fibrotic Cytokines.

Cardiac fibrosis having been found in senescence-prone mice displaying age-related diastolic dysfunction, signaling pathways that might contribute to the fibrotic response were investigated. TGF-β is a potent pro-fibrotic cytokine that influences the development of cardiac fibrosis by promoting cellular events such as increased collagen synthesis and decreased protease expression (Lijnen et al., 2000, Induction of cardiac fibrosis by transforming growth factor-beta(1). Mol Genet Metab 71: 418-435; Lim & Zhu, 2006, Role of transforming growth factor-beta in the progression of heart failure. Cell Mol Life Sci 63: 2584-2596). Moreover, connective tissue growth factor (CTGF) is induced by TGF-β and acts synergistically with TGF-β to promote deposition of extracellular matrix proteins (Chen et al., 2000, CTGF expression is induced by TGF- beta in cardiac fibroblasts and cardiac myocytes: a potential role in heart fibrosis. J Mol Cell Cardiol 32: 1805-1819; Leask, 2007,TGFbeta, cardiac fibroblasts, and the fibrotic response. Cardiovasc Res 74: 207-212; Lim & Zhu, 2006, Id.). These pro-fibrotic cytokines were known to be capable of converting fibroblasts into myofibroblasts, which express alpha smooth-muscle actin (α-SMA) and synthesize collagen, promoting the fibrotic process (Lijnen et al., 2000, Id.). Thus, expression of TGF-β and CTGF (as reflected in increased α-SMA expression) was assessed in SAMP8 as compared with control SAMR1 mice using Western blotting. In these experiments, Western blotting was performed as described in Nisbet et al. (2009, The role of NADPH oxidase in chronic intermittent hypoxia-induced pulmonary hypertension in mice. Am J Respir Cell Mol Biol 40: 601-609, incorporated by reference). Briefly, ILeft ventricular heart tissue homogenates were prepared in a buffer containing 20 mM Tris (pH 7.4), 2.5 mM EDTA, 1% triton X-100, 1% deoxycholic acid, 0.1% SDS, 100 mM NaCl, 10 mM NaF, and 1 mM Na₃VO₄ and quantified using the BCA protein assay according to the manufacturer's instructions (Thermo Scientific, Rockford, Ill.). Heart samples (40 μg protein per lane) were run on a 10% SDS-PAGE gel (Invitrogen, Carlsbad, Calif.) for 90 minutes at 150V then transferred to a polyvinylidene fluoride (PVDF) membrane. Membranes were blocked for 30 minutes in 5% nonfat dry milk and probed with primary antibody (1:1,000) specific to TGF-β (Santa Cruz Biotechnology) or α-smooth muscle actin (Thermo Fisher Scientific, Fremont, Calif.) on a rocking platform overnight at 4° C. Membranes were washed, then incubated with horseradish peroxidase-conjugated secondary antibody (Jackson ImmunoResearch), and detected using the SuperSignal West Pico peroxide and luminol enhancer solution (Thermo Scientific, Rockford, Ill.). Membranes were imaged, photographed, and quantified using the BioRad ChemiDoc system (Hercules, Calif.). Proteins of interest were normalized to cdk4 content.

The results of these experiments are shown in FIGS. 6A, 6B and 6C. Cardiac gene expression of TGF-β and CTGF were increased in SAMP8 mice compared to SAMR1 controls (FIGS. 6A and 6B). Consistent with increased collagen disposition, α-SMA protein expression was increased in SAMP8 mice compared to controls, suggesting a conversion of fibroblasts into myofibroblasts (FIG. 6C).

These results indicated that age-related diastolic dysfunction was associated with cytokine-stimulated increases in extracellular matrix components, including collagens, fibronectin and α-SMA, and that amelioration of fibrotic processes that characterize age-related diastolic dysfunction could be achieved as set forth herein.

It should be understood that the foregoing disclosure emphasizes certain specific embodiments of the invention and that all modifications or alternatives equivalent thereto are within the spirit and scope of the invention as set forth in the appended claims.

We claim:

1. A method of treating age-associated diastolic dysfunction in the absence of systolic dysfunction in a human or comprising administering to the human or animal an anti-fibrotic agent in a therapeutically effective amount.

2. The method of claim 1, wherein the diastolic dysfunction comprises increased cardiac fibrosis.

3. The method of claim 1, wherein the diastolic dysfunction is characterized by abnormal proliferation of extracellular matrix components.

4. The method of claim 3, wherein the human or animal exhibits myocardial or left or right ventricular diastolic stiffness.

5. The method of any one of the preceding claims, wherein the anti-fibrotic agent is at least one of an inhibitor of transforming growth factor-β (TGF-β), an inhibitor of connective tissue growth factor (CTGF), or an inhibitor of a SMAD protein.

6. The method of claim 5, wherein the TGF-β inhibitor inhibits expression of TGF-β.

7. The method of claim 5, wherein the CTGF inhibitor inhibits expression of CTGF.

8. The method of claim 5, wherein the SMAD protein inhibitor inhibits expression of SMAD protein.

9. The method of claim 1, wherein the anti-fibrotic agent is neither human growth hormone (GH) nor a TGF-β neutralizing antibody.

10. The method of claim 1, wherein the subject is a human.

11. A method of treating a human or animal for age-associated diastolic dysfunction in the absence of systolic dysfunction, comprising administering to the human or animal an anti-fibrotic agent, provided that the anti-fibrotic agent is neither human growth hormone (GH) or a TGF-β neutralizing antibody.

12. The method of claim 11 wherein the age-associated diastolic dysfunction is present in the human or animal in the absence of cardiac oxidative stress.

13. The method of claim 11, wherein the age-associated diastolic dysfunction is characterized by increased cardiac fibrosis.

14. The method of claim 11, wherein the age-associated diastolic dysfunction comprises or is characterized by abnormal proliferation of the extracellular matrix components.

15. The method of claim 11, wherein the subject exhibits myocardial and left or right ventricular diastolic stiffness.

16. The method of claim 11, wherein the anti-fibrotic agent is at least one of an inhibitor of transforming growth factor-β (TGF-β), an inhibitor of connective tissue growth factor (CTGF), or an inhibitor of a SMAD protein.

17. The method of claim 16, wherein the TGF-β inhibitor inhibits TGF-β expression.

18. The method of claim 16, wherein the CTGF inhibitor inhibits CTGF expression.

19. The method of claim 16, wherein the SMAD protein inhibitor inhibits expression of SMAD protein.

20. The method of claim 11, wherein the subject is a human.

21. A pharmaceutical composition for use in a method in any of the preceding claims, comprising at least one of an inhibitor of transforming growth factor-β (TGF-β), an inhibitor of connective tissue growth factor (CTGF), or an inhibitor of a SMAD protein and a pharmaceutically acceptable carrier, diluent or excipient.

22. A kit for practicing the method of claims 1 through 20 comprising a pharmaceutical composition comprising at least one of an inhibitor of transforming growth factor-β (TGF-β), an inhibitor of connective tissue growth factor (CTGF), or an inhibitor of a SMAD protein and a pharmaceutically acceptable carrier, diluent or excipient and optionally instructions for the use thereof.
